(19) United States
(12) Patent Application Publication
(10) Pub. No.: US 2025/0369966 A1
(43) Pub. Date: Dec. 4, 2025

(54) System and Method for Analyzing Image of Cell and/or Tissue, and Computer Readable Medium Thereof

(71) Applicant: Anivance AI Corporation, Hsinchu County (TW)

(72) Inventors: Guan-Yu Chen, Hsinchu County (TW); Ren-Hao Xie, Hsinchu County (TW); Shiue-Luen Chen, Yilan County (TW)

(73) Assignee: National Yang Ming Chiao Tung University, Hsinchu City (TW)

(21) Appl. No.: 18/679,492

(22) Filed: May 31, 2024

(30) Foreign Application Priority Data
May 31, 2024 (TW) 113120196

(51) Int. Cl. G01N 33/569; B01L 3/00; C12M 1/12; C12M 1/34; C12M 3/00; G01N 21/64; G06V 10/82; G06V 20/69; G06V 20/70

(52) U.S. Cl. CPC G01N 33/56966; B01L 3/502715; C12M 21/08; C12M 25/02; C12M 41/46; G01N 21/6428; G06V 10/82; G06V 20/693; G06V 20/695; G06V 20/698; G06V 20/70; B01L 2200/16; B01L 2300/0654; G01N 2021/6439; G06V 2201/03

(57) ABSTRACT

A system and method for analyzing image of cell and/or tissue are provided. The system may carry an organ-on-a-chip having the cell and/or the tissue, and may have an image capturing module and an analysis module. The image capturing module may be used to capture the image of the cell and/or the tissue from the organ-on-a-chip. The analysis module may be used to extract image feature from the image of the cell and/or the tissue, and label a classification of the cell and/or the tissue according to the image feature.

### BACKGROUND OF THE INVENTION

### 1. Field of the Invention

The invention is related to image identification technique, in particular to a system, method and computer readable medium for analyzing image of cell and/or tissue.

### 2. Description of the Prior Art

Organ-on-a-Chip (OoC) is a type of rising micro-cell culture technique that is particularly efficient in replicating structure and function of human organs. Currently, function and quality of cell and/or tissue cultured in OoC can only be observed under the the following conditions: (I) the cell and/or tissue is at a culture age sufficient to express cell biology status; (II) the cell and/or the tissue is at a culture age sufficient to react to stimulation of staining agents and/or biomarkers; and (III) research personnel possess accurate analyzing skills to observe the cell and/or the tissue through instruments. Therefore, the operation for culturing the cell and/or the tissue on OoC may be time-consuming and has a high error rate since the above conditions are hard to meet.

Therefore, there is an urgent need in the industry for a system, method and computer readable medium for analyzing image of cell and/or tissue to identify function and quality of cell and/or tissue during initial stage of culturing on OoC and cut down cost, time and human error for culturing cell and/or tissue.

### SUMMARY OF THE INVENTION

In at least one embodiment of the present invention, a system for analyzing image of cell and/or tissue may be disposed with an organ-on-a-chip having the cell and/or the tissue and include an image capturing module and an analysis module coupled to the image capturing module. The image capturing module may be used to capture an image of the cell and/or the tissue from the organ-on-a-chip. The analysis module may be used to extract an image feature from the image of the cell and/or the tissue, and label a classification of the cell and/or the tissue according to the image feature.

In at least one embodiment of the present invention, a method for analyzing image of cell and/or tissue may include providing an organ-on-a-chip having the cell and/or the tissue, an image capturing module capturing an image of the cell and/or the tissue from the organ-on-a-chip, an analysis module extracting an image feature from the image of the cell and/or the tissue, and the analysis module labeling a classification of the cell and/or the tissue according to the image feature.

In at least one embodiment of the present invention, a computer readable medium may store a computer executable instruction which, when being executed, causes the method for analyzing image of cell and/or tissue to be implemented.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of components of a system for analyzing image of cells and/or tissue.

FIG. 2 is a schematic diagram of components of an organ-on-a-chip.

FIG. 3 is a schematic diagram of process for culturing the cell and/or the tissue.

FIG. 4A to FIG. 4D are schematic diagrams of various types of image of air-liquid interface cell culture of HSAEC.

FIG. 5 is a schematic diagram of image of immersed cell culture and image of air-liquid interface cell culture of the cell and/or the tissue of HSAEC.

FIG. 6 is a schematic diagram of procedures for the model building module training the CNN of the analysis module.

FIG. 7A to FIG. 7E are schematic diagrams of result from augmentation pre-processing.

FIG. 8A and FIG. 8B are schematic diagrams of application results of the CNN using Score-CAM.

FIG. 9A and FIG. 9B are schematic diagrams of training results of ResNet.

### DETAILED DESCRIPTION

The following describes the implementation of the present disclosure with examples. Those skilled in the art can easily understand the spirit, advantages and effects of the present disclosure from the content disclosed in this specification. However, the embodiments set forth herein are not intended to limit the present disclosure, and the present disclosure can also be implemented or applied by other different embodiments, and the details set forth herein can also be based on different viewpoints and applications. Various changes or modifications can be made without departing from the spirit of the present disclosure.

The features such as a ratio, structure, and dimension shown in drawings accompanied with the present disclosure are simply used to cooperate with the contents disclosed herein for those skilled in the art to read and understand the present disclosure, rather than to limit the scope of implementation of the present disclosure. Thus, in the case that does not affect the purpose of the present disclosure and the effect brought by the present disclosure, any change in proportional relationships, structural modification, or dimensional adjustment should fall within the scope of the technical contents disclosed herein.

When “comprising,” “including,” or “having” an element described herein, unless otherwise specified, other elements, components, structures, regions, parts, devices, systems, steps, or connection relationships and other requirements may be further included, rather than excluding those other requirements. In addition, unless otherwise specified, the singular forms “a” and “the” used herein also include plural forms, and the terms “or” and “and/or” used herein are interchangeable.

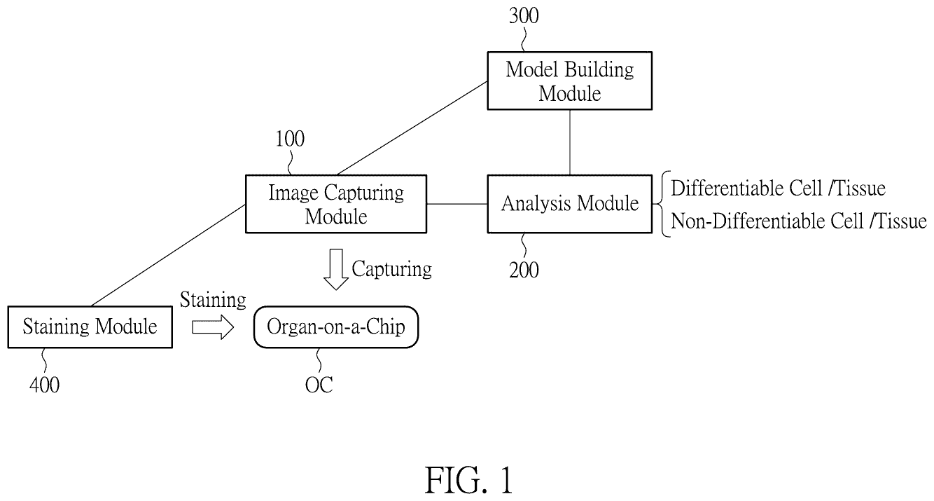

FIG. 1 is a schematic diagram of components of a system for analyzing image of cells and/or tissue. The system may include an image capturing module 100, an analysis module 200, a model building module 300, and a staining module 400. The above components may be coupled to each other via any suitable wired or wireless means. Additionally, the system for analyzing image of cell and/or tissue may be disposed with an organ-on-a-chip (OoC) OC. The OoC OC may be used to culture the cell and/or the tissue.

The image capturing module 100 may be realized as any suitable microscopic imaging device for capturing image of the cell and/or the tissue on the OoC OC. In the embodiment, the image of the cell and/or the tissue may include an image of immersed cell culture and an image of air-liquid interface cell culture according to different culture stages in the OoC OC. Furthermore, the image of immersed cell culture may be a bright-field cell image, and the image of air-liquid interface cell culture may be a fluorescence-stained cell images.

The analysis module 200 may be coupled with the image capturing module 100 and may be realized as any one or more of a mainframe computer, a personal computer, a tablet computer, a mobile device, a cloud storage device, an application, a server, a virtual machine, or any combination thereof. The analysis module 200 may be equipped with a convolutional neural network (CNN) and may be used to extract an image feature from the image of the cell and/or the tissue and label a classification of the cell and/or the tissue according to the image feature. The classification of the cell and/or the tissue may include differentiable cell and/or tissue and non-differentiable cell and/or tissue, among other classifications related to cell biology status of the cell and/or the tissue. Additionally, the analysis module 200 may be used to analyze the image of immersed cell culture during labeling of the classification of the cell and/or the tissue.

The model building module 300 may be coupled with the image capturing module 100 and the analysis module 200, and may be realized as one or more of a mainframe computer, a personal computer, a tablet computer, a mobile device, a cloud storage device, an application, a server, a virtual machine, or any combination thereof. The model building module 300 may be used to preprocess the image of the cell and/or the tissue, establish a model building dataset, and train the analysis module 200 to label the classification of the cell and/or the tissue according to the image feature using the model building dataset. The analysis module 200 and the model building module 300 may also be realized as a sole integrated component or formed by various components with different functions in charge of respective details for analyzing the image of the cell and/or the tissue.

The staining module 400 may be coupled with the image capturing module 100, may be realized as any suitable staining instrument, and may be used to perform fluorescence staining for the cell and/or the tissue on the OoC OC. The fluorescence staining for the cell and/or the tissue may allow the image capturing module 100 to capture the image of air-liquid interface cell culture using the stained cell and/or stained tissue. The image of air-liquid interface cell culture may be used to observe correlation between the image of air-liquid interface cell culture and the image of immersed cell culture and thereby establish the model building dataset.

Also provided is a method for analyzing image of cell and/or tissue, which may be realized through the image capturing module 100, the analysis module 200, the model building module 300, and the staining module 400 as mentioned above. The method may include capturing the image of the cell and/or the tissue, extracting the image feature from the image of the cell and/or the tissue, labeling the classification of the cell and/or the tissue according to the image feature, and training the CNN of the analysis module 200.

Further provided is a computer-readable medium storing a computer executable instruction which, when being executed, causes the method for analyzing image of cell and/or tissue to be implemented.

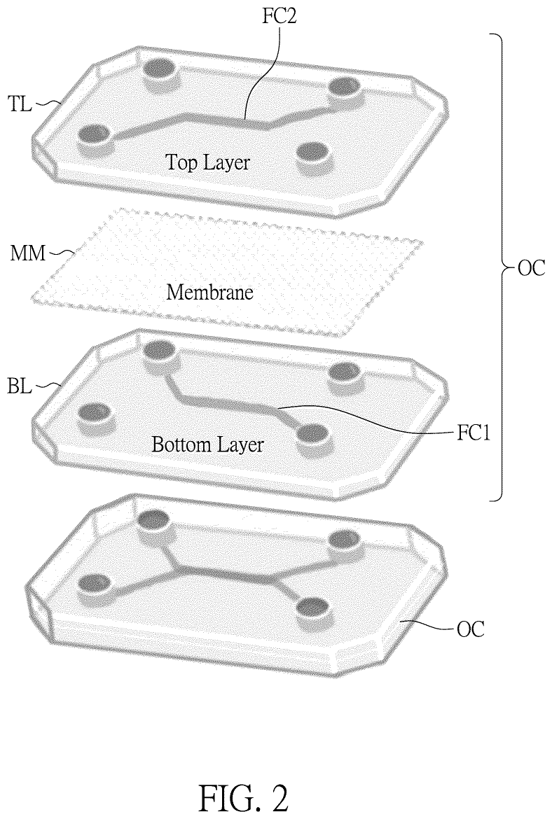

FIG. 2 is a schematic diagram of components of the OoC OC. The OoC may include a bottom layer BL, a membrane MM, and a top layer TL. The bottom layer BL and the top layer TL may include a flow channel FC1 and a flow channel FC2, respectively. The membrane MM may be a porous membrane formed from polyethylene terephthalate (PET) and may be used for seeding the cell and/or the tissue. The flow channels FC1 and FC2 may be microchannels formed from polycarbonate (PC) plastic sheets and arranged parallel to each other, and may be used for circulating and supplying culture medium for the cell and/or the tissue seeded on the membrane MM. The cell and/or the tissue cultured in the OoC OC may be cell and/or tissue of respiratory system, such as human small airway epithelial cells (HSAEC). The embodiments described hereafter will explain the process for culturing the cell and/or the tissue of HSAEC using the OoC OC

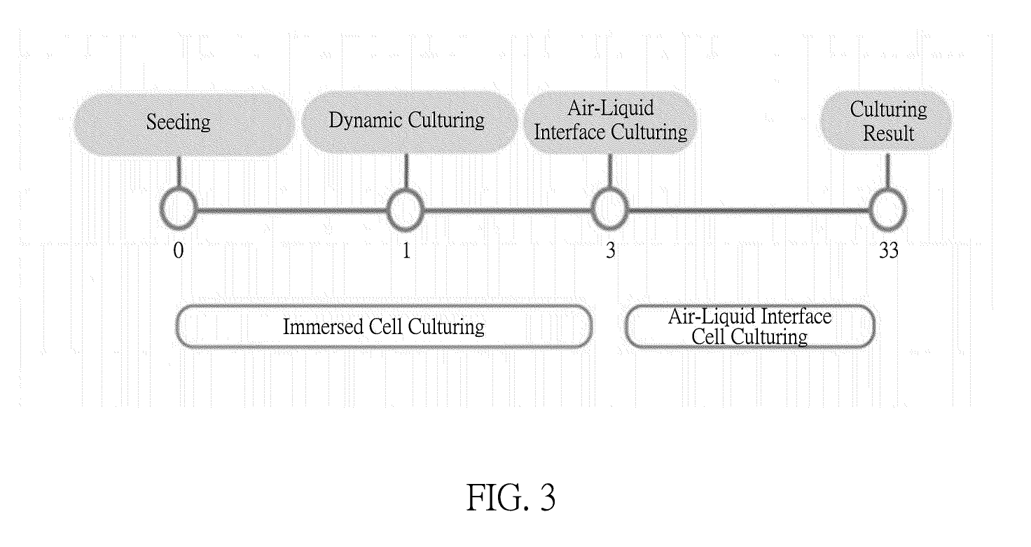

FIG. 3 is a schematic diagram of the process for culturing the cell and/or the tissue using the OoC OC shown in FIG. 2. In FIG. 3, the node and corresponding number on the time axis represent the time elapsed (e.g., Day X, where X is an integer) since the HSAEC is seeded on the membrane MM of the OoC OC. On Day 0, the HSAEC is seeded on the membrane MM, the bottom layer BL and the top layer TL of the OoC OC are attached to each other, and the flow channels FC1 and FC2 may circulate the culture medium for dynamically culturing the HSAEC in an immersed condition. On Day 3, the HSAEC on the membrane MM is fully converged, the top layer TL of the OoC OC may be removed to form an air-liquid interface (ALI), and the HSAEC may thus be cultured under the ALI until differentiation takes place. On Day 33, as the HSAEC has reached the peak of differentiation, the cell and/or the tissue on the OoC OC may be harvested to confirm cell biology status of the cell and/or the tissue. In other words, the image of immersed cell culture may be a bright-field cell image taken during period of the cell and/or the tissue being cultured in the immersed culturing condition, the image of air-liquid interface cell culture may be a fluorescent staining cell image taken during period of the cell and/or the tissue being cultured in the ALI culturing condition and the cell and/or the tissue is fluorescent stained. Moreover, the image of air-liquid interface cell culture may be used to reflect cell biology status of the cell and/or the tissue after cultivation and may therefore act as ground truth of model building dataset for validating accuracy of the analysis module 200 while labeling the classification of the cell and/or the tissue according to the image of immersed cell culture. Additionally, the durations of the immersed culturing period and the ALI culturing period are not limited to Day 0 to Day 3 and Day 3 to Day 33, respectively, and can be adjusted to any length according to type of the cell and/or the tissue and culturing environment set by the OoC OC.

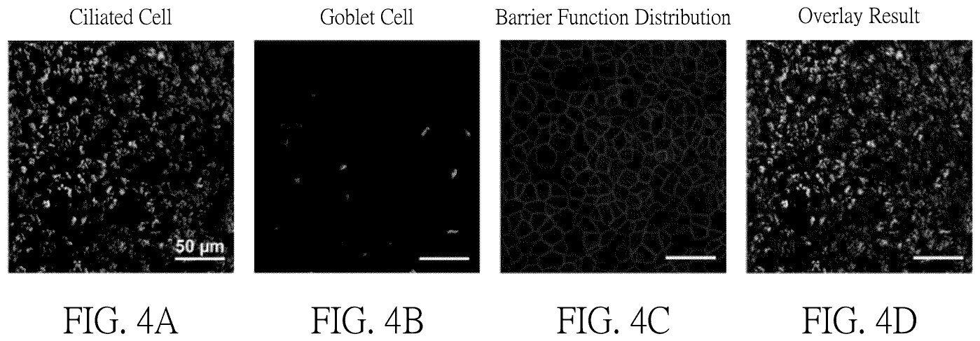

FIG. 4A to FIG. 4D are schematic diagrams of various types of image of air-liquid interface cell culture of HSAEC, where different cell biology statuses may be observed through different staining agents and biomarkers provided by the staining module 400. FIG. 4A shows the fluorescent stained image of differentiated ciliated cells of HSAEC after anti-acetylated tubulin (AC-Tubulin) staining, and the stained area by AC-Tubulin is shown as yellow. FIG. 4B shows the fluorescent stained image of differentiated goblet cells of HSAEC after mucin 5B (MUC5B) staining, and the stained area of MUC5B is shown as green. FIG. 4C shows the fluorescent stained image of barrier function distribution of differentiated HSAEC after zonula occludens-1 (ZO-1) staining, and the stained area of ZO-1 is shown as red. FIG. 4D shows overlay of the fluorescent stained images of HSAEC after AC-Tubulin staining, MUC5B staining, ZO-1 staining and 4′, 6-diamidino-2-phenylindole (DAPI) counterstaining, and the stained area of DAPI is shown as blue. From here, the image of air-liquid interface cell culture may be used to observe different cell biology statuses under influences of different staining agents and biomarkers, train the analysis module 200 to predict the corresponding cell biology status of the cell and/or the tissue during the immersed culturing period early on according to correlation between different types of image of air-liquid interface cell culture and the image of immersed cell culture. The embodiments described hereafter will explain the process of the model building module 300 establishing a model building dataset using the fluorescence-stained image of HSAEC at day 33 of culturing and after ZO-1 staining, and the analysis module 200 training to predict whether HSAEC is differentiable cell and/or differentiable tissue according to the image of immersed cell culture.

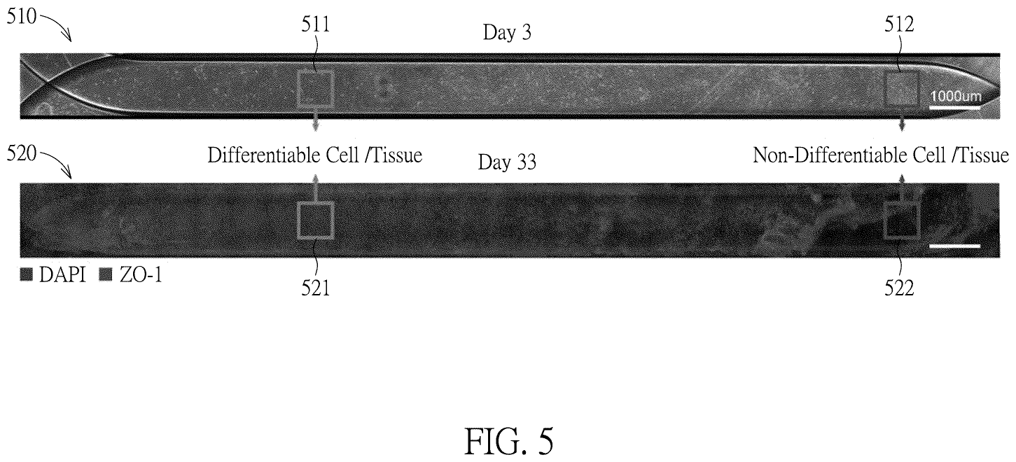

FIG. 5 is a schematic diagram of the image of immersed cell culture 510 and the image of air-liquid interface cell culture 520 of the cell and/or the tissue of HSAEC from the same OoC OC. The image of immersed cell culture 510 is captured from immersed culturing period (e.g., at day 3) of HSAEC culturing, and is a bright-field cell image. The image of air-liquid interface cell culture 520 is captured from ALI culturing period (e.g., at day 33) of HSAEC culturing, and is a fluorescent staining cell image resulted from ZO-1 staining and DAPI counterstaining. The area 521 of the image of air-liquid interface cell culture 520 may be regarded as ZO-1 area, where good cell barrier function of the cell and/or tissue may prompt cell barrier area to appear. Consequently, the area 511 of the image of immersed cell culture 51 corresponding to the area 521 may be labeled as “differentiable cell and/or differentiable tissue” by the model building module 300 and act as part of the model building dataset. On the other hand, the area 522 of the image of air-liquid interface cell culture 520 may be regarded as non-ZO-1 area, where the lack of differentiation ability of the cell and/or the tissue may prompt fibrosis area to appear. Consequently, the area 512 of the image of immersed cell culture 510 corresponding to the area 522 may be labeled as “non-differentiable cell and/or non-differentiable tissue” by the model building module 300 and act as part of the model building dataset.

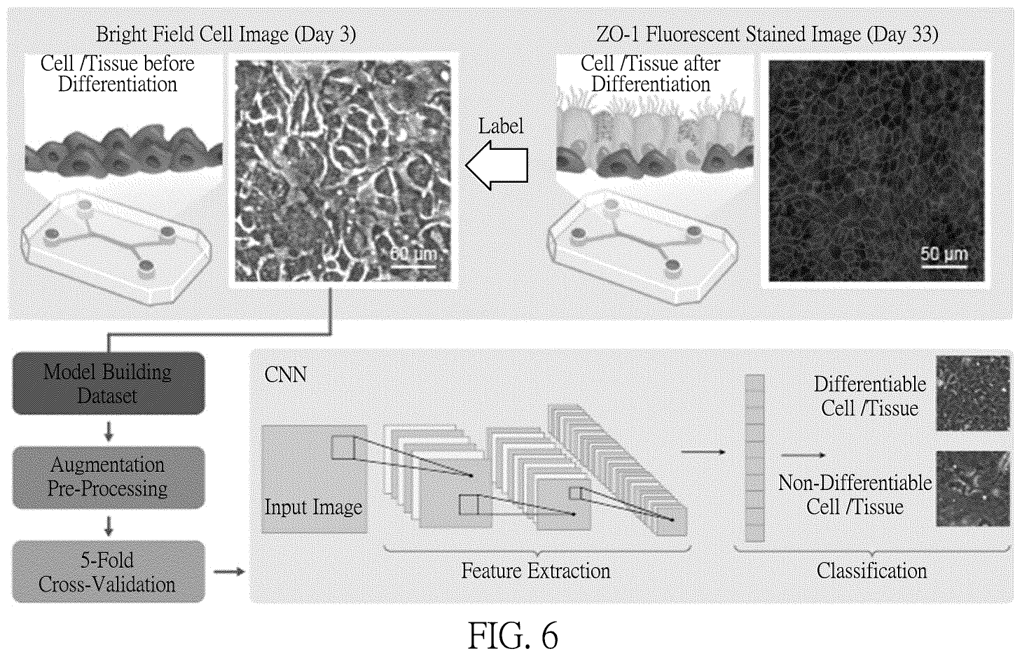

FIG. 6 is a schematic diagram of procedures for the model building module 300 training the CNN of the analysis module 200, where the procedures may be sequentially understood by following the direction indicated by the arrows, and the CNN is trained according to embodiment of determining classification of “differentiable cell and/or differentiable tissue” or “non-differentiable cell and/or non-differentiable tissue” for the cell and/or the tissue of HSAEC. However, it should be understood that the same procedures may also be applicable to other types and/or classification of cell and/or tissue in the respiratory system.

First, the model building module 300 may, with reference to conditions shown in FIG. 5, generate a model building dataset according to the correlation between the image of air-liquid interface cell culture and the image of immersed cell culture. The process for generating the model building dataset may include: labeling the ZO-1 area and the non-ZO-1 area of the differentiated cell and/or the differentiated tissue in the image of air-liquid interface cell culture; overlapping the image of air-liquid interface cell culture with the corresponding image of immersed cell culture; and segmenting the areas of the image of immersed cell culture corresponding to the ZO-1 area and/or the non-ZO-1 area into equal sizes to form the model building dataset. From here, each piece of data in the model building dataset may be labeled as one of “differentiable cell and/or differentiable tissue” and “non-differentiable cell and/or non-differentiable tissue” according to the ZO-1 area and non-ZO-1 area, respectively. In some embodiments, the predetermined size for segmenting the image of immersed cell culture may be 224 pixel×224 pixels.

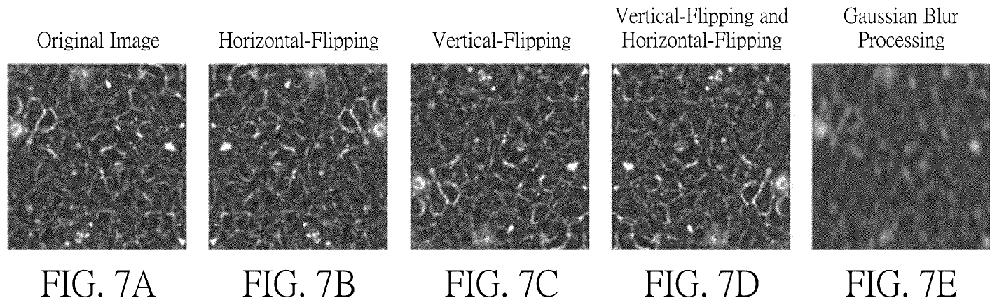

Next, the model building module 300 may perform augmentation pre-processing on the model building dataset to enhance generalization ability and accuracy of the CNN. The augmentation pre-processing may be understood through FIG. 7A to FIG. 7E: FIG. 7A is related to one piece of data of the modeling building dataset, which is an original image with size of 224 pixel×224 pixel; FIG. 7B is related to a first augmented data corresponding to the original image after horizontal-flipping; FIG. 7C is related to a second augmented data corresponding to the original image after vertical-flipping; FIG. 7D is related to a third augmented data corresponding to the original image after vertical-flipping and horizontal-flipping; and FIG. 7E is related to a fourth augmented data corresponding to the original image after Gaussian blur processing.

Next, returning to FIG. 6, the model building module 300 may train the CNN of the analysis module 200 with the model building dataset after augmentation pre-processing. In here, a 5-fold cross-validation is utilized to validate accuracy of the analysis module 200 being trained by the model building dataset. For example, the model building dataset may be divided into five subsets, the CNN may be trained via five learning iterations, each learning iteration may rotate one of the five subsets to act as training set and the remaining four of the five subsets to act as validation set, an average accuracy from the five learning iterations may be determined as training result of the CNN. The above practice may ensure arrangements of the training set and the validation set to be representative of the model building dataset as a whole.

Finally, the CNN may be applied to operational environment after accuracy thereof is confirmed through 5-fold cross-validation. Therefore, the analysis module 200 may receive image input from the image acquisition module 100, extract the image feature from the image and classify the cell and/or the tissue in the image as either a “differentiable cell and/or differentiable tissue” or a “non-differentiable cell and/or non-differentiable tissue.”

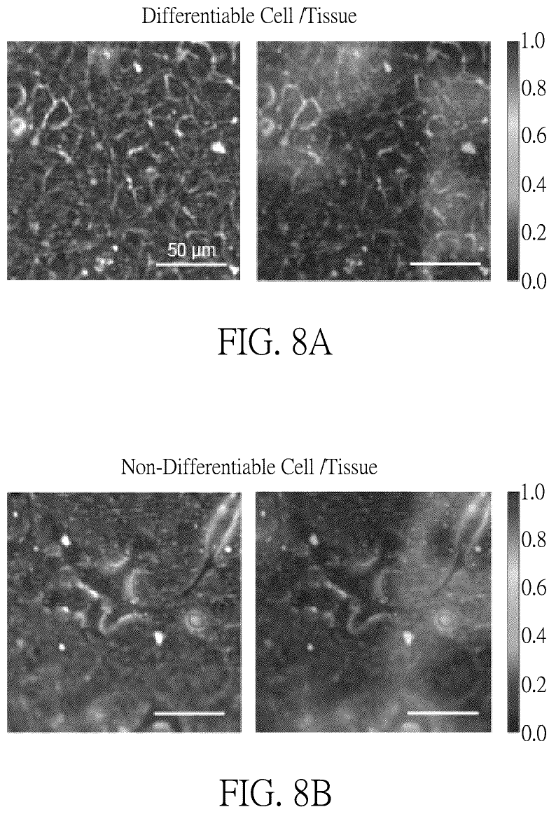

The CNN of the analysis module 200 may utilize score-weighted class activation mapping (Score-CAM) technique to visualize the result of the analysis module 200 labeling the image of the cell and/or the tissue. The application results of the CNN using Score-CAM are shown in FIG. 8A and FIG. 8B. Score-CAM may process the extracted image feature using Softmax score as weight to eliminate dependence of the extract image feature towards unstable gradients and display the image feature in form of focus hotspot, where focus hotspot may be marked in red and non-focus hotspot may be marked in blue.

FIG. 8A is a schematic diagram of respective image of the cell and/or the tissue before and after labeling by the analysis module 200 using Score-CAM for differentiable cell and/or differentiable tissue. The left image is an original image of immersed cell culture regarding the differentiable cell and/or the differentiable tissue of HSAEC, which is input into the CNN of the analysis module 200. The right image shows visualized classification of “differentiable cell and/or differentiable tissue” labeled on the original image of immersed cell culture after the Score-CAM capturing the cell barrier area to act as focus hotspot.

FIG. 8B is a schematic diagram of respective image of the cell and/or the tissue before and after labeling by the analysis module 200 using Score-CAM for non-differentiable cell and/or non-differentiable tissue. The left image is an original image of immersed cell culture regarding the non-differentiable cell and/or the non-differentiable tissue of HSAEC, which is input into the CNN of the analysis module 200. The right image shows visualized classification of “non-differentiable cell and/or non-differentiable tissue” labeled on the original image of immersed cell culture after the Score-CAM capturing the cell fibrosis area to act as focus hotspot.

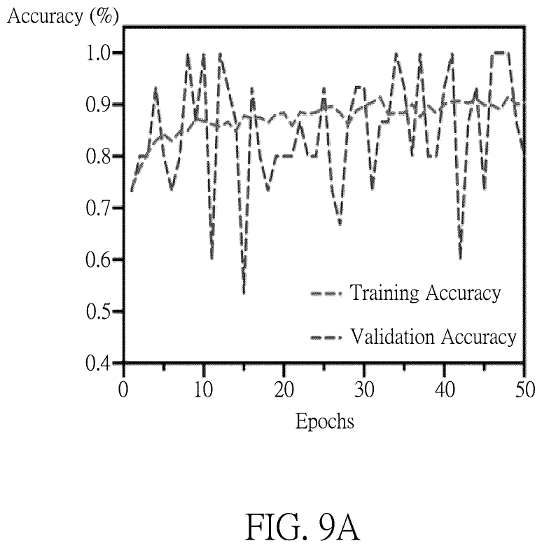

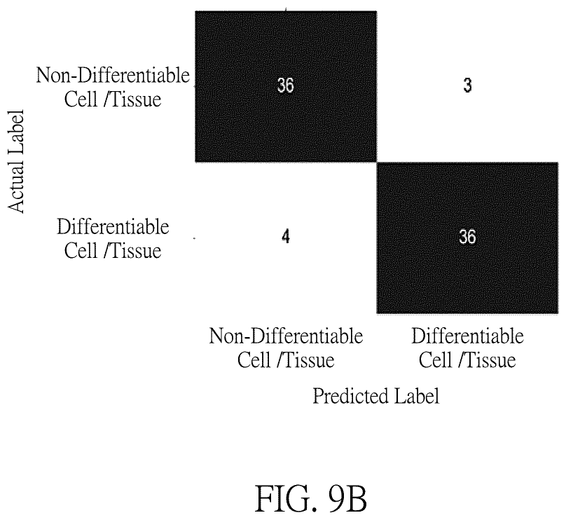

In this embodiment, the CNN of the analysis module 200 may be implemented as any one of ResNet, GoogLeNet, VGG16, or AlexNet, or any other suitable CNNs. FIG. 9A and FIG. 9B are schematic diagrams of training results of the analysis module 200 using ResNet correctly classifying “differentiable cell and/or differentiable tissue” or “non-differentiable cell and/or non-differentiable tissue” from the image of immersed cell culture regarding the cell and/or the tissue of HSAEC.

FIG. 9A is a schematic diagram of line chart of training accuracy and validation accuracy of the ResNet after 50 epochs of training. In here, prediction accuracy of the ResNet for determining differentiation ability of the cell and/or tissue at day 33 according to the image of immersed cell culture of HSAEC cultured on the day 3 is approximately at 89.14%.

FIG. 9B is a schematic diagram of a confusion matrix of result of the trained ResNet labeling classification of the cell and/or the tissue of HSAEC according to image of immersed cell culture. In here, superior accuracy of the ResNet allows predicted labels and actual labels to converge at top-left and bottom-right corners of the confusion matrix, respectively. That is, the analysis module 200 may correctly predict and label non-differentiable cell and/or non-differentiable tissue as classification of “non-differentiable cell and/or non-differentiable tissue” and differentiable cell and/or differentiable tissue as classification of “differentiable cell and/or differentiable tissue”.

Based on the above, the system, the method, and the computer-readable medium of the present invention may be applied to organ-on-a-chip culture technique requiring both immersed cell culture and air-liquid interface culture. Therefore, cell biology status of cell and/or tissue to be shown during air-liquid interface culture may be determined during immersed cell culture of the cell and/or the tissue, and successful culturing of the cell and/or the tissue culture may be predicted as early as possible. Additionally, waiting time for determining cell biology status of the cell and/or the tissue at later stages of culture may be omitted and cost for culturing the cell and/or the tissue may be reduced.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A system for analyzing an image of a cell and/or a tissue, configured to carry an organ-on-a-chip having the cell and/or the tissue, comprising:
an image capturing module, configured to capture the image of the cell and/or the tissue from the organ-on-a-chip; and
an analysis module, coupled to the image capturing module and configured to:
extract an image feature from the image of the cell and/or the tissue; and
label a classification of the cell and/or the tissue according to the image feature.

2. The system of claim 1, wherein:
the cell and/or the tissue is a cell and/or a tissue of a respiratory system; and
the image of the cell and/or the tissue comprises an image of immersed cell culture, wherein the image of immersed cell culture is a bright-field cell image.

3. The system of claim 1, wherein:
the image of the cell and/or the tissue comprises a image of immersed cell culture;
the analysis module is equipped with a convolutional neural network;
the convolutional neural network is configured to capture a focus hotspot from the image of immersed cell culture via a score-weighted class activation mapping technique, thereby capturing the image feature according to the focus hotspot;
when the focus hotspot identifies cell barrier area in the image of immersed cell culture, the analysis module labels the classification of the cell and/or the tissue corresponding to the cell barrier area in the image of immersed cell culture as a differentiable cell and/or a differentiable tissue; and
when the focus hotspot identifies cell fibrosis area in the image of immersed cell culture, the analysis module labels the classification of the cell and/or the tissue corresponding to the cell fibrosis area in the image of immersed cell culture as a non-differentiable cell and/or a non-differentiable tissue.

4. The system of claim 2, wherein:
the image of the cell and/or the tissue comprises a image of air-liquid interface cell culture; and
the system further comprises a model building module coupled with the analysis module and the image capturing module, and the model building module is configured to:
label the classification of the image of immersed cell culture according to correlation between the image of air-liquid interface cell culture and the image of immersed cell culture;
perform augmentation pre-processing on the image of immersed cell culture;
establish a model building dataset according to the image of immersed cell culture processed by the augmentation pre-processing; and
train the analysis module according to the model building dataset.

5. The system of claim 4, wherein:
the model building dataset comprises:
first augmented data corresponding to the image of immersed cell culture after horizontal-flipping;
second augmented data corresponding to the image of immersed cell culture after vertical-flipping;
the third augmented data corresponding to the image of immersed cell culture after vertical-flipping and horizontal-flipping; and/or
the fourth augmented data corresponding to the image of immersed cell culture after Gaussian blur processing; and
the model building module is further configured to:
perform 5-fold cross-validation to validate accuracy of the analysis module being trained by the model building dataset.

6. The system of claim 4, further comprising:
a staining module coupled to the image capturing module and configured to perform fluorescent staining for the cells and/or the tissue on the organ-on-a-chip, wherein
a capture time of the image of air-liquid interface cell culture is later than a capture time of the image of immersed cell culture, and
the image of air-liquid interface cell culture is a fluorescent staining cell image.

7. The system of claim 6, wherein the model building module is further configured to:
when the image of air-liquid interface cell culture shows the cell and/or the tissue after fluorescent staining forms a zonula occludens-1 area, label the classification of the cell and/or the tissue having the zonula occludens-1 area in the image of immersed cell culture as a differentiable cell and/or a differentiable tissue; and
when the image of air-liquid interface cell culture shows the cell and/or the tissue after fluorescent staining forms a non-zonula occludens-1 area, label the classification of the cell and/or the tissue having the non-zonula occludens-1 area in the image of immersed cell culture as a non-differentiable cell and/or a non-differentiable tissue.

8. A method for analyzing an image of a cell and/or a tissue, comprising:
providing an organ-on-a-chip having the cell and/or the tissue;
an image capturing module capturing the image of the cell and/or the tissue from the organ-on-a-chip;
an analysis module extracting an image feature from the image of the cell and/or the tissue; and
the analysis module labeling a classification of the cell and/or the tissue according to the image feature.

9. The method of claim 8, wherein:
the cell and/or the tissue is a cell and/or a tissue of a respiratory system; and
the image of the cell and/or the tissue comprises an image of immersed cell culture, wherein the image of immersed cell culture is a bright-field cell image.

10. The method of claim 8, wherein:
the image of the cell and/or the tissue comprises an image of immersed cell culture;
the analysis module is equipped with a convolutional neural network; and
the analysis module labeling the classification of the cell and/or the tissue according to the image feature comprises:
the convolutional neural network capturing a focus hotspot from the image of immersed cell culture via a score-weighted class activation mapping technique, thereby capturing the image feature according to the focus hotspot;
when the focus hotspot identifies cell barrier area in the image of immersed cell culture, the analysis module labeling the classification of the cell and/or the tissue corresponding to the cell barrier area in the image of immersed cell culture as a differentiable cell and/or a differentiable tissue; and
when the focus hotspot identifies cell fibrosis area in the image of immersed cell culture, the analysis module labeling the classification of the cell and/or the tissue corresponding to the cell fibrosis area in the image of immersed cell culture as a non-differentiable cell and/or a non-differentiable tissue.

11. The method of claim 9, wherein:
the image of the cell and/or the tissue comprises an image of air-liquid interface cell culture; and
the method further comprises:
a model building module labeling the classification of the image of immersed cell culture according to correlation between the image of air-liquid interface cell culture and the image of immersed cell culture;
the model building module performing augmentation pre-processing on the image of immersed cell culture;
the model building module establishing a model building dataset according to the image of immersed cell culture processed by the augmentation pre-processing; and
the model building module training the analysis module according to the model building dataset.

12. The method of claim 11, wherein:
the model building module performing the augmentation pre-processing on the image of immersed cell culture comprises:
performing horizontal-flipping on the image of immersed cell culture to obtain first augmented data;
performing vertical-flipping on the image of immersed cell culture to obtain second augmented data;
performing vertical-flipping and horizontal-flipping on the image of immersed cell culture to obtain third augmented data; and/or
performing Gaussian blur processing on the image of immersed cell culture to obtain fourth augmented data; and
the model building module training the analysis module according to the model building dataset comprises:
performing 5-fold cross-validation to validate accuracy of the analysis module being trained by the model building dataset.

13. The method of claim 11, further comprising:
a staining module performing fluorescent staining for the cells and/or the tissue on the organ-on-a-chip, wherein
a capture time of the image of air-liquid interface cell culture is later than a capture time of the image of immersed cell culture, and
the image of air-liquid interface cell culture is a fluorescent staining cell image.

14. The method of claim 13, wherein the model building module labeling the classification of the image of immersed cell culture according to correlation between the image of air-liquid interface cell culture and the image of immersed cell culture comprises:
when the image of air-liquid interface cell culture shows the cell and/or the tissue after fluorescent staining forms a zonula occludens-1 area, the model building module labeling the classification of the cell and/or the tissue having the zonula occludens-1 area in the image of the immersed cell culture as a differentiable cell and/or a differentiable tissue; and
when the image of air-liquid interface cell culture shows the cell and/or the tissue after fluorescent staining forms a non-zonula occludens-1 area, the model building module labeling the classification of the cell and/or the tissue having the non-zonula occludens-1 area in the image of immersed cell culture as a non-differentiable cell and/or a non-differentiable tissue.

15. A computer readable medium storing a computer executable instruction which, when being executed, causes the method of claim 8 to be implemented.
